(19) United States
(12) Patent Application Publication
(10) Pub. No.: US 2022/0192970 A1
(43) Pub. Date: Jun. 23, 2022

(54) NATURAL HAIR CARE TREATMENT AND METHOD OF USING

(71) Applicant: Tatianna Byrd, Dickinson, ND (US)

(72) Inventor: Tatianna Byrd, Dickinson, ND (US)

(21) Appl. No.: 17/537,370

(22) Filed: Nov. 29, 2021

Related U.S. Application Data
(60) Provisional application No. 63/119,322, filed on Nov. 30, 2020.

(51) Int. Cl. A61K 8/9739; A61Q 5/00; A61K 8/9789; A61K 8/92; A61K 8/67

(52) U.S. Cl. CPC A61K 8/9739; A61Q 5/00; A61K 8/678; A61K 8/922; A61K 8/9789

(57) ABSTRACT

A composition for the treatment of hair including sea moss, and aloe vera, wherein the sea moss and aloe vera together comprise at least 75% by weight of the total weight of the composition.

This application claims priority to a United States Provisional Patent Application filed on Nov. 30, 2020, in the United States Patent and Trademark Office and assigned Ser. No. 63/119,332, the entire contents of which are incorporated herein by reference.

### FIELD OF INVENTION

The exemplary embodiments relate to a hair care product composition, and more particularly, to an all-natural hair care product composition that has been designed and developed to treat and promote healthy human hair growth. The exemplary embodiments also relate to an all-natural scalp and hair ointment that has been designed and developed to treat dry hair and promote growth.

The exemplary embodiments also relate to a plant based organic leave-in hair care product composition comprising sea moss, aloe vera, vitamin E, apricot oil, avocado oil, and grapeseed. In alternative exemplary embodiments, the exemplary hair care product composition may further include a fragrance.

Alternative exemplary embodiments also relate to a plant based organic scalp ointment composition comprising mango butter, shea butter, coco butter, castor oil, aloe vera, fractured or fractionated coconut oil, avocado oil, rosemary, lavender, and peppermint.

### BACKGROUND

Many commercially available haircare products are synthesized with chemicals and reagents that are proven to be caustic and harmful to the human body. Use of these products removes natural oils from hair and scalp.

Therefore, what is desired are plant based all-natural hair care products that promote hair growth without risking damage to the hair or harm to the human body.

### SUMMARY OF THE INVENTION

Certain of the foregoing and related aspects are readily attained according to the present general inventive concept by providing a composition for the treatment of hair, including sea moss, and aloe vera, wherein the sea moss and aloe vera together comprise at least 75% by weight of the total weight of the composition.

The composition may further include vitamin E, apricot oil, avocado oil, and grapeseed.

The composition may further include at least one fragrance.

The sea moss may be present in the composition in an amount ranging from 15-70% by weight of the total weight of the composition.

The aloe vera may be present in the composition in an amount ranging from 15-70% by weight of the total weight of the composition.

The vitamin E may be present in the composition in an amount ranging from 0.5-10% by weight of the total weight of the composition.

The apricot oil may be present in the composition in an amount ranging from 0.5-10% by weight of the total weight of the composition.

The avocado oil may be present in the composition in an amount ranging from 0.5-10% by weight of the total weight of the composition.

The grapeseed may be present in the composition in an amount ranging from 0.5-10% by weight of the total weight of the composition.

The fragrance may be present in the composition in an amount ranging from 0.5-10% by weight of the total weight of the composition.

The composition may further include mango butter, shea butter, coco butter, castor oil, aloe vera, fractionated coconut oil, avocado oil, rosemary, lavender, and peppermint.

Certain of the foregoing and related aspects are readily attained according to the present general inventive concept by also providing a composition for the treatment of hair, including mango butter, shea butter, and coco butter, wherein the mango butter, shea butter, and coco butter together comprise at least 10% by weight of the total weight of the composition.

The composition may further include castor oil, aloe vera, fractionated coconut oil,

avocado oil, rosemary, lavender, and peppermint.

The mango butter may be present in the composition in an amount ranging from 5-45% by weight of the total weight of the composition.

The shea butter may be present in the composition in an amount ranging from 5-45% by weight of the total weight of the composition.

The coco butter may be present in the composition in an amount ranging from 5-45% by weight of the total weight of the composition.

The castor oil may be present in the composition in an amount ranging from 5-65% by weight of the total weight of the composition.

The aloe vera may be present in the composition in an amount ranging from 5-65% by weight of the total weight of the composition.

The fractionated coconut oil may be present in the composition in an amount ranging from 5-65% by weight of the total weight of the composition.

The rosemary may be present in the composition in an amount ranging from 1-10% by weight of the total weight of the composition, the lavender is present in the composition in an amount ranging from 1-25% by weight of the total weight of the composition, and the peppermint is present in the composition in an amount ranging from 1-25% by weight of the total weight of the composition.

The composition may further include sea moss, aloe vera, vitamin E, apricot oil, avocado oil, grapeseed, and at least one fragrance.

Certain of the foregoing and related aspects are readily attained according to the present general inventive concept by also providing a method of preparing the composition according to a present general inventive concept including combining the sea moss and the aloe vera, adding the vitamin E, apricot oil, avocado oil, and grapeseed; and blending the composition.

Wherein in (c), the composition may be blended for at least 60 seconds, and the method further include d) prior to (c), adding at least one fragrance.

Certain of the foregoing and related aspects are readily attained according to the present general inventive concept by also providing a method of preparing the composition according to the present general inventive concept includes melting the mango butter, shea butter, and coco butter; adding the castor oil, aloe vera, fractionated coconut oil, avocado oil, rosemary, lavender, and peppermint; cooling the composition; permitting the composition to reach ambient temperature; and blending the composition.

Wherein in (a), the mango butter, the shea butter, and the coco butter may be melted at a temperature between 122° F. and 132° F., in (c), the composition may be cooled at a temperature below 45° F. for a duration of time between 60 minutes and 90 minutes, in (d), the composition may be permitted to reach ambient temperature for at least 60 minutes, and in (e), the composition may be blended for at least 5 minutes.

In addition, the device and method according to the present inventive concept provides plant based all-natural hair care products that promote hair growth without risking damage to the hair or harm to the human body.

### BRIEF DESCRIPTIONS OF THE DRAWINGS

These and or other aspects of the exemplary embodiments will become apparent and more readily appreciated from the following description thereof, taken in conjunction with the accompanying drawings of which:

FIG. 1 is a schematic overview of an exemplary composition.

FIG. 2 is a schematic overview of an exemplary method of preparing an exemplary composition.

FIG. 3 is a schematic overview of an exemplary method of preparing an alternative exemplary composition.

### DESCRIPTION OF INVENTION

Reference will now be made in detail to the exemplary embodiments and methods, some of which are illustrated in the accompanying drawings, wherein like reference numerals refer to the like elements throughout. The exemplary embodiments and methods are described below, occasionally with reference to the figures.

It should be understood that all percentages used herein are calculated by weight unless otherwise indicated. Further, all percentages are calculated based on a total composition weight of 100% unless otherwise indicated.

The exemplary embodiments disclosed herein are used in the fields of cosmetics, dermatology, treating, managing, and protecting keratinous fibers such as hair, as well treating, managing, and protecting skin, for example, skin of the scalp. The exemplary embodiments are applied to hair and skin as a topical application. The exemplary embodiments provide comfort, moisturization, stability, as well as provide damage resistance to hair and skin. Additionally, the exemplary embodiments are applicable for hair cleaning, hair styling, hair moisturizing, and hair treatment. However, as can be appreciated, these applications of the embodiments are merely exemplary, and the exemplary embodiments may be used in other applications as well.

The exemplary embodiments also protect hair and skin from damage sustained from exposure to heat, such as from blow drying, contact with a curling or crimping iron, and contact with a straightening iron or flat iron. Further, the exemplary embodiments cause only minimal fading of a previously applied hair color when applied to the hair and scalp. However, the exemplary embodiments provide additional benefits as well.

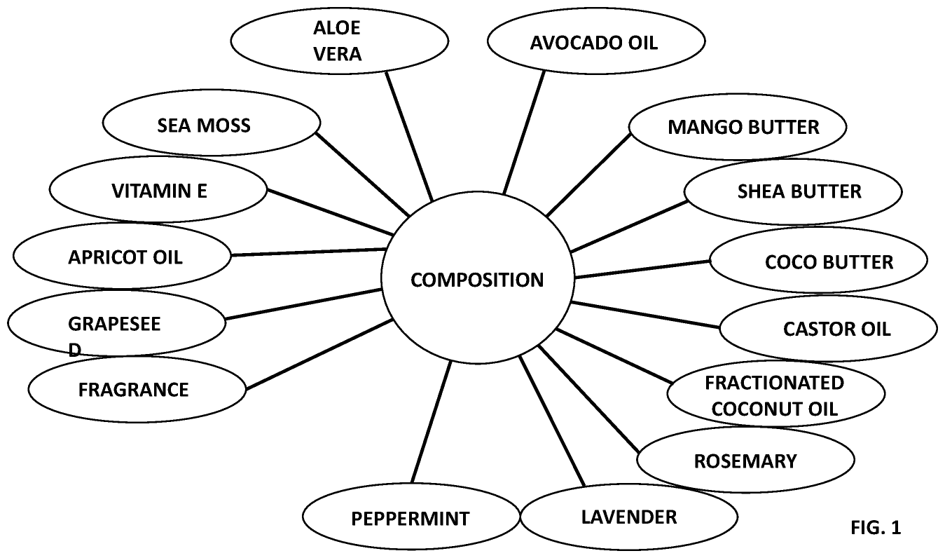

In exemplary embodiments of the all-natural hair care product and organic scalp ointment shown in FIG. 1, the exemplary compositions comprise at least one botanical compound. The exemplary botanical compounds include, without limitation, Chondrus crispus (“sea moss”), Tocopherol or Alpha-tocopherol acetate (ATA) (“vitamin E oil”), Persea americana or Persea gratissima (“avocado oil”), Mangifera Indica (“mango butter”), Vitellaria paradoxa (“shea butter”), Theobroma Cocoa (“coco or cocoa butter”), Ricinus communis (“castor oil”), Cocos nucifera (“coconut oil or fractionated coconut oil or fractured coconut oil”), Lavandula angustifolia (“lavender”), Mentha piperita (“peppermint”), Salvia rosmarinus (“rosemary”), aloe barbadensis leaf extract (“aloe vera”), Prunus armeniaca kernel extract (“apricot oil”), and palmitoyl grape seed extract or Vittis vinifera seed extract (“grapeseed”). In alternative exemplary embodiments, the exemplary compositions further include Helianthus annuus (“sunflower seed oil”), Santalum album extract or oil (“sandalwood oil”), Phellodendron amurense bark extract or oil (“bark oil”), and Hordeum distichon extract or oil (“barley oil”). Of course, as should be understood, these elements of the exemplary compositions are merely exemplary, and in other exemplary compositions, other elements may be used.

It should be understood that as used herein, “butter” used in combination with any of the disclosed botanical compound means a fat and/or oil extract of a plant, seed, or fruit, and having emollient properties as well as a melting point near human body temperature. However, as should also be understood, although the exemplary botanical compounds disclosed herein are or have been described as being present in the composition in the form of a butter or oil, in alternative exemplary embodiments, the botanical compounds are present in the composition in other forms as well. For example, the botanical compounds are present in alternative exemplary compositions in the form of extracts or pure oils, in diluted extracts or oils, or in extracts or oils including unavoidable impurities or trace elements. In further alternative exemplary embodiments, the exemplary compositions include a tolerable threshold of unavoidable impurities or trace elements. It should be further understood that the unavoidable impurities or trace elements are not accounted for separately in the total composition weight, due to their generally infinitesimal presence. As should be further understood, the exemplary compositions disclosed herein may further include fillers such as water, or any other filler known in the cosmetics, hair care, and skin care industries.

In alternative exemplary embodiments, the exemplary composition further includes at least one fragrance. The exemplary fragrance includes any fragrance or smell desired by the preparer of the composition. For example, the exemplary fragrance includes any floral note, oriental note, woody note, or any fresh note. As can be appreciated, these notes include floral, soft floral, floral oriental, soft oriental, oriental, woody oriental, woody, mossy wood, dry wood, aromatic, citrus, water, green, and fruity. However, it should be understood that any fragrance may be used, either now known or later developed including commercially available fragrances, perfumes, or colognes, and including any combination of the above disclosed notes.

In some exemplary embodiments, the exemplary composition comprises a plant based organic leave-in hair care product composition comprising sea moss, aloe vera, vitamin E oil, apricot oil, avocado oil, and grapeseed oil. In exemplary embodiments, the sea moss and aloe vera comprise the predominant botanical compounds in the exemplary compositions, meaning that these botanical compounds are present in the exemplary compositions in amounts greater than other elements of the compositions. In fact, in some exemplary embodiments, the sea moss and aloe vera comprise between at least 50% and 92% by weight of the total weight of the composition. However, as can be appreciated, these elements and respective amounts of the composition are merely exemplary, and in other exemplary compositions, other elements and amounts may be used.

In an exemplary embodiment, the exemplary composition comprises a plant based organic leave-in hair care product containing, sea moss present in the composition in an amount ranging between 40 and 46% by weight of the total weight of the composition, aloe vera present in the composition in an amount ranging between 40 and 46% by weight of the total weight of the composition, vitamin E oil present in the composition in an amount ranging between 0.5 and 1.6% by weight of the total weight of the composition, apricot oil present in the composition in an amount ranging between 0.5 and 1.6% by weight of the total weight of the composition, avocado oil present in the composition in an amount ranging between 0.5 and 1.6% by weight of the total weight of the composition, grapeseed oil present in the composition in an amount ranging between 0.5 and 1.6% by weight of the total weight of the composition, and at least one fragrance present in the composition in an amount ranging between 0.5 and 1.6% by weight of the total weight of the composition. This exemplary composition is provided in Table 1 below.

TABLE 1

| | Exemplary Composition |
|---|---|
| Ingredients | (ranges by weight % of the total composition) |
| Sea moss | 40-46 |
| Aloe vera | 40-46 |
| Vitamin E | 0.5-1.6 |
| Apricot Oil | 0.5-1.6 |
| Avocado Oil | 0.5-1.6 |
| Grapeseed | 0.5-1.6 |
| Fragrance | 0.5-1.6 |

However, as can be appreciated, this composition arrangement is merely exemplary, and in other embodiments, other arrangements may be used.

In an exemplary alternative embodiment, the exemplary composition comprises a plant based organic scalp ointment composition containing mango butter, shea butter, coco butter, castor oil, aloe vera oil, fractionated coconut oil, avocado oil, rosemary, lavender, and peppermint. In exemplary embodiments, the mango butter, shea butter, and coco butter comprise the predominant botanical compounds in the exemplary alternative compositions, meaning that these botanical compounds are present in the alternative compositions in amounts greater than other elements of the alternative embodiments. In fact, in some alternative exemplary embodiments, the mango butter, shea butter, and coco butter comprise between 25% and 65% by weight of the total weight of the composition. However, as should be understood, these elements and amounts of the alternative compositions are merely exemplary, and in other compositions, other elements and amounts may be used.

In one alternative exemplary embodiment, the mango butter is present in the composition in an amount ranging between 10 and 20% by weight of the total weight of the composition, the shea butter is present in the composition in an amount ranging between 10 and 20% by weight of the total weight of the composition, the coco butter is present in the composition in an amount ranging between 10 and 20% by weight of the total weight of the composition, the castor oil is present in the composition in an amount ranging between 5 and 15% by weight of the total weight of the composition, the aloe vera is present in the composition in an amount ranging between 5 and 15% by weight of the total weight of the composition, the fractured or fractionated coconut oil is present in the composition in an amount ranging between 5 and 15% by weight of the total weight of the composition, the avocado oil is present in the composition in an amount ranging between 5 and 10% by weight of the total weight of the composition, the rosemary is present in the composition in an amount ranging between 1 and 5% by weight of the total weight of the composition, the lavender is present in the composition in an amount ranging between 1 and 5% by weight of the total weight of the composition, and the peppermint is present in the composition in an amount ranging between 1 and 5% by weight of the total weight of the composition. This alternative exemplary embodiment is provided below in Table 2.

TABLE 2

| | Exemplary Composition |
|---|---|
| Ingredients | (ranges by weight % of the total composition) |
| Mango Butter | 10-20 |
| Shea Butter | 10-20 |
| Coco Butter | 10-20 |
| Castor Oil | 5-15 |
| Aloe Vera | 5-15 |
| Fractionated Coconut Oil | 5-15 |
| Avocado Oil | 5-10 |
| Rosemary | 1-5 |
| Lavender | 1-5 |
| Peppermint | 1-5 |

Of course, as should be understood, these arrangements are merely exemplary, and in other embodiments, other arrangements may be used.

It should be understood that alterations may be made to the exemplary compositions disclosed herein without departing from the scope of this disclosure. For example, in some exemplary embodiments, the at least one fragrance may be forgone, or certain elements of the exemplary composition may be foregone to provide hypoallergenic embodiments. For example, if one of the exemplary elements listed above is known to cause allergic reactions in a significant number of users, the element may be foregone, or a counteracting supplemental or a substitute element may be used in addition or in place thereof. Of course, these alterations of the composition are merely exemplary, and other alterations may be made on a needed basis.

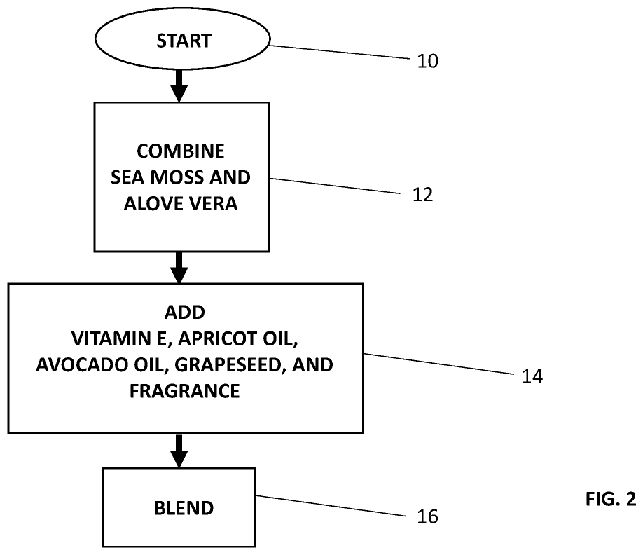

Now will be described exemplary methods of preparing the exemplary embodiments disclosed herein. An exemplary method of preparing an exemplary composition for the plant based organic hair care product is represented by the schematic shown in FIG. 2. As shown in FIG. 2, the exemplary method for preparing the exemplary composition begins at step 10. At step 12, the sea moss and the aloe vera are combined. At step 14, the vitamin E oil, avocado oil, apricot oil, grapeseed, and at least one fragrance are added to the combination of sea moss and aloe vera. At step 16, the mixture produced from steps 12 and 14 is blended. It should be understood that the term “blend” or “blended” as used herein includes stirring, mixing, or otherwise combining or distributing to form a homogenous mixture. However, as can be appreciated, this method is merely exemplary, and in other methods, other steps may be taken to produce the exemplary composition.

In an exemplary method of preparing the exemplary plant based organic hair care product composition according to Table 1, the 40-46% sea moss and 40-46% aloe vera are first combined. Next, the 0.5-1.6% vitamin E, 0.5-1.6% apricot oil, 0.5-1.6% avocado oil, 0.5-1.6% grapeseed, and 0.5-1.6% fragrance are added to the mixture. The mixture is then blended for at least 60 seconds, but preferably for about 75 seconds. However, this method is merely exemplary, and in other embodiments, other methods or process steps may be used.

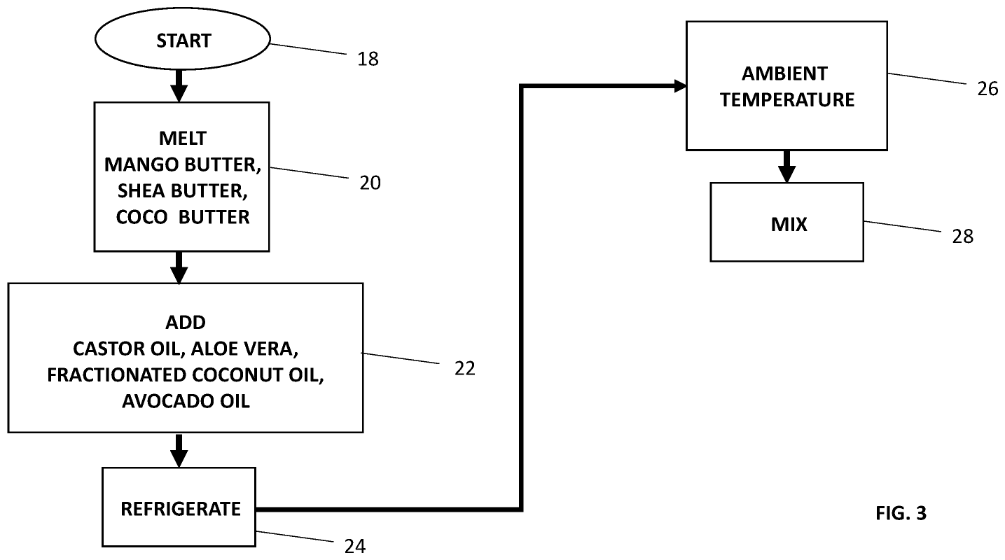

An exemplary method of preparing the alternative exemplary composition used as a scalp ointment and hair care product is represented by the schematic shown in FIG. 3. As shown in FIG. 3, the exemplary method for preparing the alternative exemplary composition begins at step 18. At step 20, the mango butter, shea butter, and cocoa butter are melted at a temperature ranging between 122° and 132° F. At step 22, the castor oil, aloe vera, fractionated coconut oil, and avocado oil are added to the mixture produced during step 20. At step 24, the mixture produced by steps 20 and 22 is cooled or refrigerated at a temperature at least below 45° F. for a duration of time of at least 45 minutes, but preferably between 60 and 90 minutes. For example, in step 24, the mixture is refrigerated in a commercially available refrigerator. At step 26, the mixture is removed from the refrigerator, and is permitted to reach room or ambient temperature. For example, at step 26, the mixture is removed from the refrigerator and permitted to sit at ambient temperature for at least 60 minutes, but preferably between 80 and 120 minutes. At step 28, after the mixture has reached ambient temperature, the mixture is then blended for at least 5 minutes, but preferably between 10 and 15 minutes. Of course, it should be understood that this method is merely exemplary, and in other exemplary methods, other process steps may be taken.

According to an alternative exemplary method of preparing the exemplary plant based organic scalp ointment composition according to Table 2, the mango butter, shea butter, and coco-butter are first melted together at a temperature between 122-132 degrees F. Next, the castor oil, aloe vera, fractured coconut oil, and avocado oil are added to the mixture. Next, the mixture is cooled or refrigerated for a duration of time between 60 and 90 minutes. The mixture is then permitted to reach room or ambient temperature for about 2 hours. Finally, the composition is mixed for about 12 minutes. As should be understood, this method is merely exemplary, and in other embodiments, other methods or process steps may be used.

As can be appreciated, the exemplary compositions presented herein are arranged to be incorporated into various cosmetic or dermatological products for hair care, skin care, hair treatment, skin treatment, as well as hair styling. For example, the exemplary composition may be incorporated into formulas for conditioning, formulas for protecting hair from heat damage, formulas for leave-in hair treatment, formulas for rinse off hair treatments, formulas for shampoos, formulas for volumizing hair, formulas for shaping hair, formulas for maintaining the shape of hair, as well as formulas for hair styling. The exemplary embodiments may be incorporated into various applicator forms including, but not limited to, gels, paste, ointments, milks, serums, creams, waxes, mousses, aerosol sprays, non-aerosol sprays, foams, lotions, or pump sprays. Additionally, the compositions may include further ingredients or elements to achieve the necessary viscosity, density, structure, texture or other properties for the above disclosed application forms. It should be understood that the exemplary compositions disclosed herein may be applied directly to the hair and scalp, applied to the hair and scalp via the use of common industry specific tools or instruments, as well as applied in either wet or dry conditions of the hair and scalp. Of course, it should be understood that these are merely exemplary applications of the exemplary compositions, and the exemplary compositions may be used in other manners as well.

Although a few exemplary embodiments have been illustrated and described, it will be appreciated by those skilled in the art that changes may be made in these exemplary embodiments and methods, and combinations of the various features and relationships and process steps of the exemplary embodiments and methods may be made without departing from the principles and spirit of this disclosure, the scope of which is defined in the appended claims and their equivalents.

What is claimed is:

1. A composition for the treatment of hair, comprising:
sea moss, and
aloe vera,
wherein the sea moss and aloe vera together comprise at least 75% by weight of the total weight of the composition.

2. The composition according to claim 1, further comprising:
vitamin E,
apricot oil,
avocado oil, and
grapeseed.

3. The composition according to claim 2, further comprising:
at least one fragrance.

4. The composition according to claim 2,
wherein the sea moss is present in the composition in an amount ranging from 40-46% by weight of the total weight of the composition.

5. The composition according to claim 2,
wherein the aloe vera is present in the composition in an amount ranging from 40-46% by weight of the total weight of the composition.

6. The composition according to claim 2,
wherein the vitamin E is present in the composition in an amount ranging from 0.5-1.6% by weight of the total weight of the composition.

7. The composition according to claim 2,
wherein the apricot oil is present in the composition in an amount ranging from 0.5-1.6% by weight of the total weight of the composition.

8. The composition according to claim 2,
wherein the avocado oil is present in the composition in an amount ranging from 0.5-1.6% by weight of the total weight of the composition.

9. The composition according to claim 2,
wherein the grapeseed is present in the composition in an amount ranging from 0.5-1.6% by weight of the total weight of the composition.

10. The composition according to claim 3,
wherein the fragrance is present in the composition in an amount ranging from 0.5-1.6% by weight of the total weight of the composition.

11. The composition according to claim 3, further comprising:
mango butter,
shea butter,
coco butter,
castor oil,
aloe vera,
fractionated coconut oil,
avocado oil,
rosemary,
lavender, and
peppermint.

12. A composition for the treatment of hair, comprising:
mango butter,
shea butter, and
coco butter,
wherein the mango butter, shea butter, and coco butter together comprise at least 25% by weight of the total weight of the composition.

13. The composition according to claim 12, further comprising:
castor oil,
aloe vera,
fractionated coconut oil,
avocado oil,
rosemary,
lavender, and
peppermint.

14. The composition according to claim 13,
wherein the mango butter is present in the composition in an amount ranging from 10-20% by weight of the total weight of the composition.

15. The composition according to claim 13,
wherein the shea butter is present in the composition in an amount ranging from 10-20% by weight of the total weight of the composition.

16. The composition according to claim 13,
wherein the coco butter is present in the composition in an amount ranging from 10-20% by weight of the total weight of the composition.

17. The composition according to claim 13,
wherein the castor oil is present in the composition in an amount ranging from 5-15% by weight of the total weight of the composition.

18. The composition according to claim 13,
wherein the aloe vera is present in the composition in an amount ranging from 5-15% by weight of the total weight of the composition.

19. The composition according to claim 13,
wherein the fractionated coconut oil is present in the composition in an amount ranging from 5-15% by weight of the total weight of the composition.

20. The composition according to claim 13,
wherein the rosemary is present in the composition in an amount ranging from 1-5% by weight of the total weight of the composition, the lavender is present in the composition in an amount ranging from 1-5% by weight of the total weight of the composition, and the peppermint is present in the composition in an amount ranging from 1-5% by weight of the total weight of the composition.
